(19) United States
(12) Patent Application Publication
(10) Pub. No.: US 2009/0180996 A1
(43) Pub. Date: Jul. 16, 2009

(54) Epigenetic modification of cell phenotype, fate and/or function by RNA transfer

(76) Inventors: Zeki Beyhan, East Lansing, MI (US); Arif Kocabas, East Lansing, MI (US); Jose Bernardo Cibelli, East Lansing, MI (US); Steven T. Suhr, Okemos, MI (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/268,095

(22) Filed: Nov. 10, 2008

Related U.S. Application Data
(60) Provisional application No. 60/986,725, filed on Nov. 9, 2007.

(51) Int. Cl. A61K 35/12; C12N 15/87; C12N 15/89; C12N 15/88; A61P 35/00; A61P 37/00; A61P 31/00; A61P 29/00; A61P 37/08

(52) U.S. Cl. 424/93.7; 435/455; 435/461; 435/458

(57) ABSTRACT

The invention relates to methods for altering the fate or differentiation status of somatic cells by RNA transfer. These methods can be used to transdifferentiate or dedifferentiate somatic cells of one phenotype or lineage into pluripotent cells or into somatic cells of a different lineage or phenotype.

### RELATED APPLICATIONS

This application claims priority to U.S. Ser. No. 60/986,725 filed on Nov. 9, 2007, which application is incorporated by reference in its entirety herein.

### FIELD OF THE INVENTION

The present invention relates to a method for changing the phenotype/fate of cells (mammalian or any other species) by transferring total or messenger RNA (mRNA) from one cell or a group of cells whose phenotype is desired to be modeled from (model cells) to another 'cell or a group of cells whose phenotype/fate is desired to be permanently modified (target cells).

The present invention may be used to provide a reproducible and renewable source of immuno-compatible human cells/tissues for transplantation based therapies. The inventive method will allow the generation of cells that are fully compatible with the patient. For example, the subject invention may obtain a simple skin biopsy and isolate primary fibroblasts (or any other cell that is easy to obtain e.g. white blood cells, keratinocytes, etc), expand them in vitro and later transdifferentiate or dedifferentiate them into desired cell populations by RNA transfection. These methods may be used to produce pluripotent cells from somatic cells by the transfer of RNA from pluripotent cells as well as the production of different somatic cells by the introduction of RNA from somatic cells such as hepatocytes, beta-cells in order to transdifferentiate one type of somatic cell into another somatic cell type.

### BACKGROUND OF THE INVENTION

As noted above, the present invention provides methods for changing the phenotype/fate of cells (mammalian or any other species) by transferring total or messenger RNA (mRNA) from one cell or a group of cells whose phenotype is desired to be modeled from (model cells) to another 'cell or a group of cells whose phenotype/fate is desired to be permanently modified (target cells).

Prior to the present invention methods of converting a somatic or differentiated cell into an embryonic stem cell had been known. Particularly, previous methods included the conversion a somatic cell into an oocyte by somatic cell nuclear transfer (SCNT) which essentially comprises the transfer of a somatic cell or the DNA or chromosomes thereof into an oocyte of the same or different species which is enucleated prior, synchronous or after the transfer of the somatic cell or DNA thereof into this oocyte.

Another previous method of converting somatic cells into a different cell lineage comprises the isolation of the cytosol from the model cell, e.g., a human oocyte and the transfer of same into the target cells, e.g., human fibroblasts.

By contrast, the present invention provides for the production of immuno-compatible human cells/tissues for transplantation based therapies and the generation of cells that are fully compatible with the patient by RNA transfer.

For example in one embodiment the present invention may be effected by obtaining a simple skin biopsy and isolating primary fibroblasts (or any other cell that is easy to obtain e.g. white blood cells, keratinocytes, etc), expanding them in vitro and later transdifferentiating or dedifferentiating them into desired cell populations by RNA transfection. Desirably, if the donor RNA is derived from a pluripotent cell such as an embryonic stem cell pluripotent cells may be derived from the recipient somatic cells, e.g., fibroblasts, as the RNA of the pluripotent cell reprograms the nucleus of the recipient somatic cell converting it into a pluripotent phenotype. Alternatively, if the RNA is obtained from another type of somatic cell, such as a hepatocyte or beta cell by way of example the RNA therefrom reprograms the nucleus of the recipient somatic cell thereby inducing transdifferentiation of the recipient somatic cell which converts the cell into the phenotype of the donor cell from which the RNA has been obtained e.g., a beta-cell or hepatocyte or other somatic cell which is desired for cell therapy.

### DESCRIPTION OF THE INVENTION AND EXEMPLARY EMBODIMENTS

The objective of the invention is to provide a method for changing the phenotype/fate of cells (mammalian or any other species) (recipient cells) by transferring total or messenger RNA (mRNA) from one cell (donor) or a group of cells whose phenotype is desired to be modeled from (model cells) to another 'cell or a group of cells whose phenotype/fate is desired to be permanently modified (target or recipient cells).

Prior to discussing the invention in more detail, the following definitions are provided. Otherwise all words and phrases in this application are to be construed by their ordinary meaning, as they would be interpreted by an ordinary skilled artisan within the context of the invention.

“Reprogramming” herein refers to the introduction of RNA from a donor cell into a recipient cell, e.g., a somatic cell, wherein the donor cell is of a different phenotype or lineage or species relative to the recipient cell under conditions whereby the donor RNA reprograms the nucleus of the recipient cell thereby converting the phenotype ore lineage of the recipient cell into that of the donor cell, e.g., a pluripotent cell or a somatic cell of a different lineage or phenotype.

“Transdifferentiation” refers to the conversion of a cell of one phenotype, e.g., a specific somatic cell into a cell of a different phenotype, e.g., a different lineage somatic cell or a pluripotent cell.

“Dedifferentiation” refers to the conversion of a somatic cell into a cell of a less differentiated phenotype, e.g., an adult or embryonic stem cell phenotype by the introduction of RNA from a pluripotent cell e.g., an embryonic stem cell, oocyte or inner cell mass cell or primordial germ cell or adult stem cell into a recipient differentiated cell, e.g., a human somatic cell.

“Pluripotent cell” refers to a cell that is capable of giving rise to all 3 cell lineages, i.e., ectoderm, endoderm and mesoderm cells.

“Multipotent” refers to a cell that is capable of giving rise to more than 1 cell lineage.

“Totipotent cell” is an undifferentiated cell such as embryonic cell such as an oocyte that is capable of giving rise to a viable offspring under appropriate conditions.

“Embryonic Stem Cell or ESC” is a cell that is capable of giving rise to all 3 lineages. ESCs may be derived from early stage embryos, umbilical cord and other embryo tissue material as well as from nuclear transfer derived embryos.

“Adult stem cell” is a cell capable of giving rise to different somatic cells of a specific lineage, e.g., immune stem cells, hematopoietic stem cells, neural stem cells, pancreatic stem cells and the like which cells are present in very few numbers in adult tissues and which cells unlike other adult somatic cells may be isolated and induced to differentiate resulting in the production of specific somatic cell lineages such as neural cells if the adult stem cell is a neural stem cell.

### DETAILED DESCRIPTION OF THE FIGURES

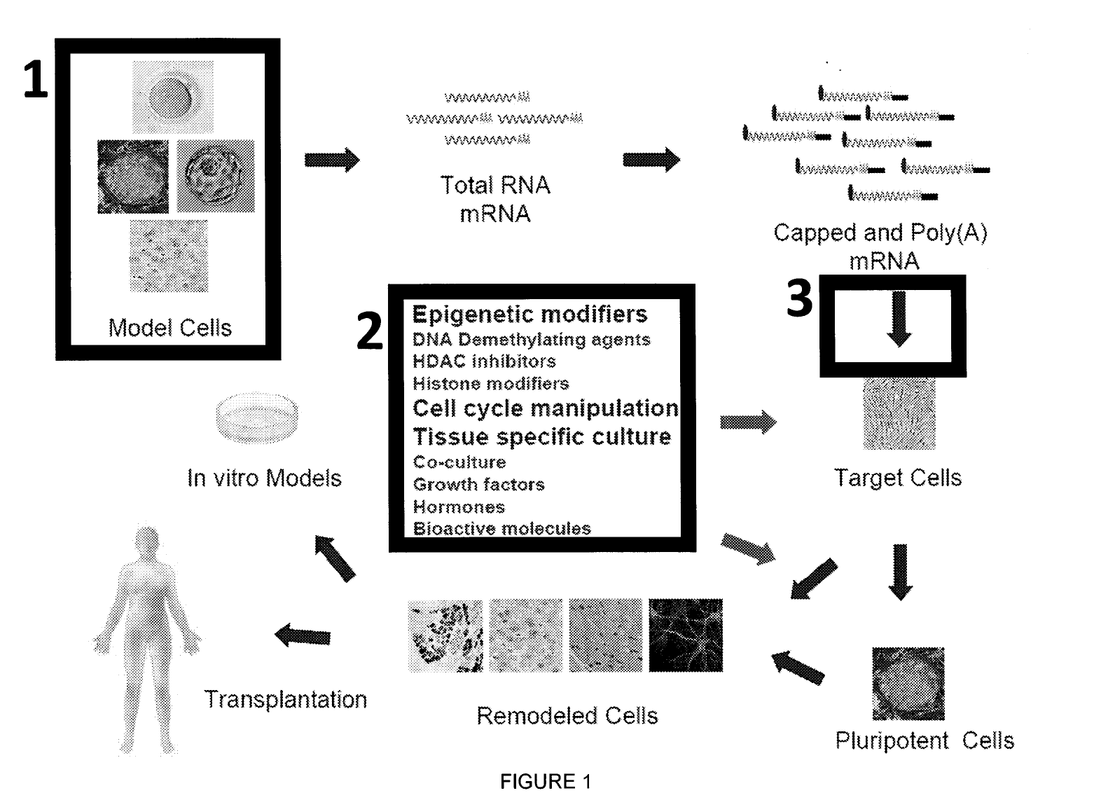

FIG. 1 depicts schematically dedifferentiation using mRNA according to the invention. This slide contains 3 yellow boxes which enumerate unique aspects of the subject dedifferentiation methods. In the square labeled #1 it can be seen that that the mRNA is isolated from different cells that they are either pluripotent (NTera, human ES, blastocysts) or are capable of turning into pluripotent cells (oocyte). Also while not specifically mentioned in the figure alternatively or in addition the invention includes further the use of mRNA from specific transcription factors to effect transdifferentiation.

In the box labeled #2 in the figure it is noted that the culture medium used in the present invention preferably may comprise additives which facilitate or promote transformation of the cells using mRNA. These additives include by way of example trichostatine, valproic acid, zebularine and 5-aza.

The box labeled #3 in FIG. 1 enumerates preferred means of delivery of the mRNA into recipient cells. These means include by way of example: electroporation, use of liposomes, and mRNA injections.

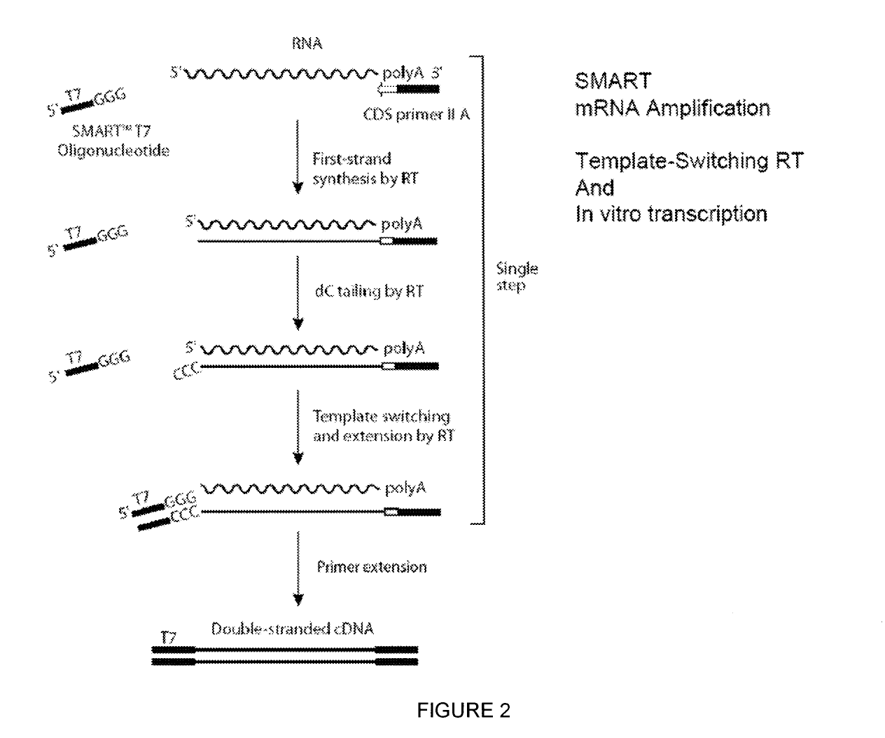

FIG. 2 depicts a preferred means of amplification of mRNA used in the present invention.

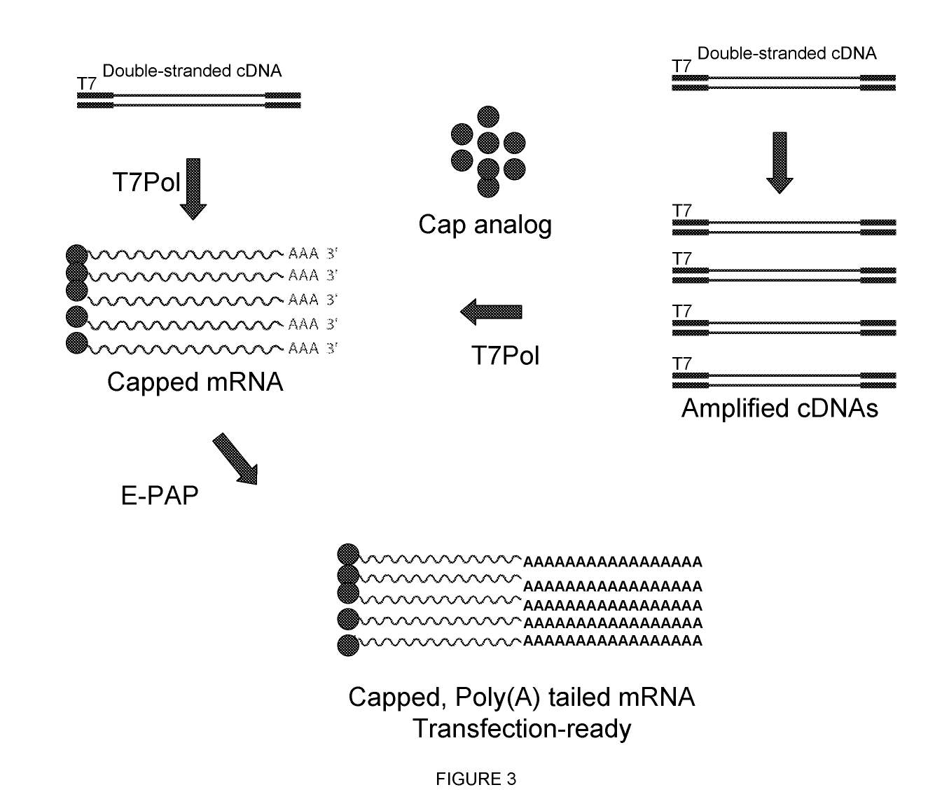

FIG. 3 also depicts schematically a preferred means of effecting mRNA amplification in the subject dedifferentiation protocols.

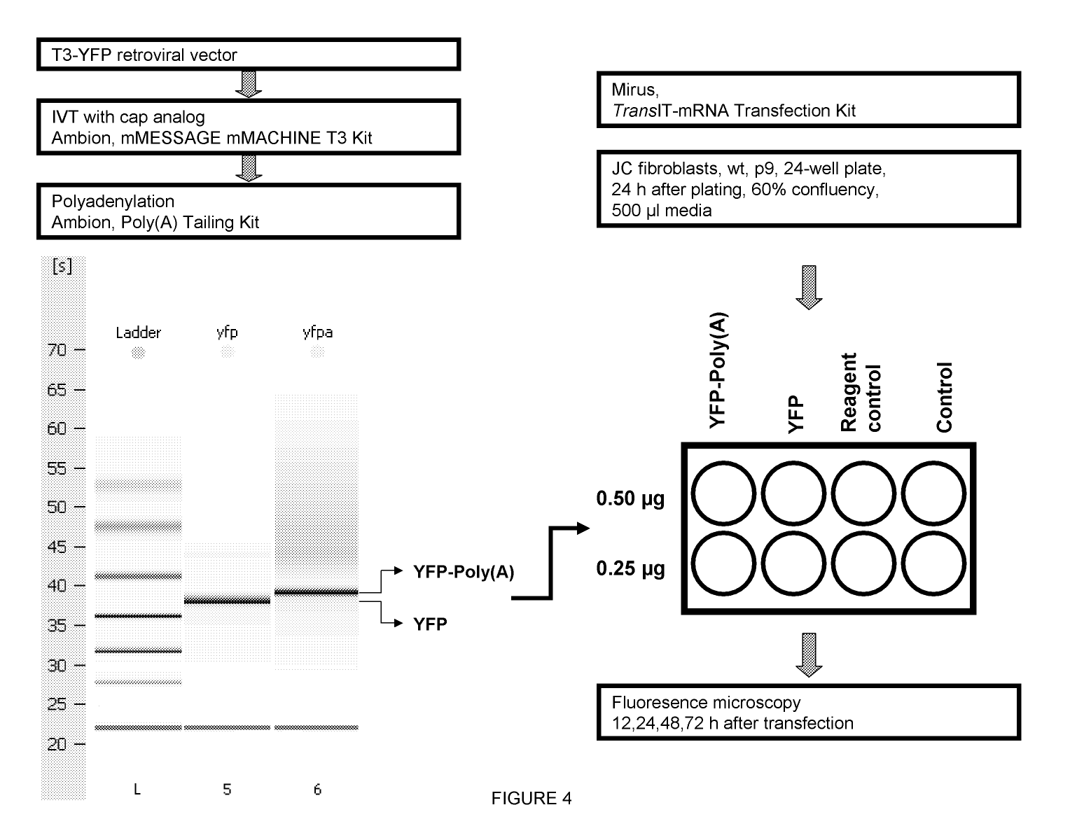

FIG. 4 contains the results of a control run showing that yellow fluorescent protein (YFP) can be in vitro transcribed with high quality and delivered to cells according to the inventive RNA transfection protocols.

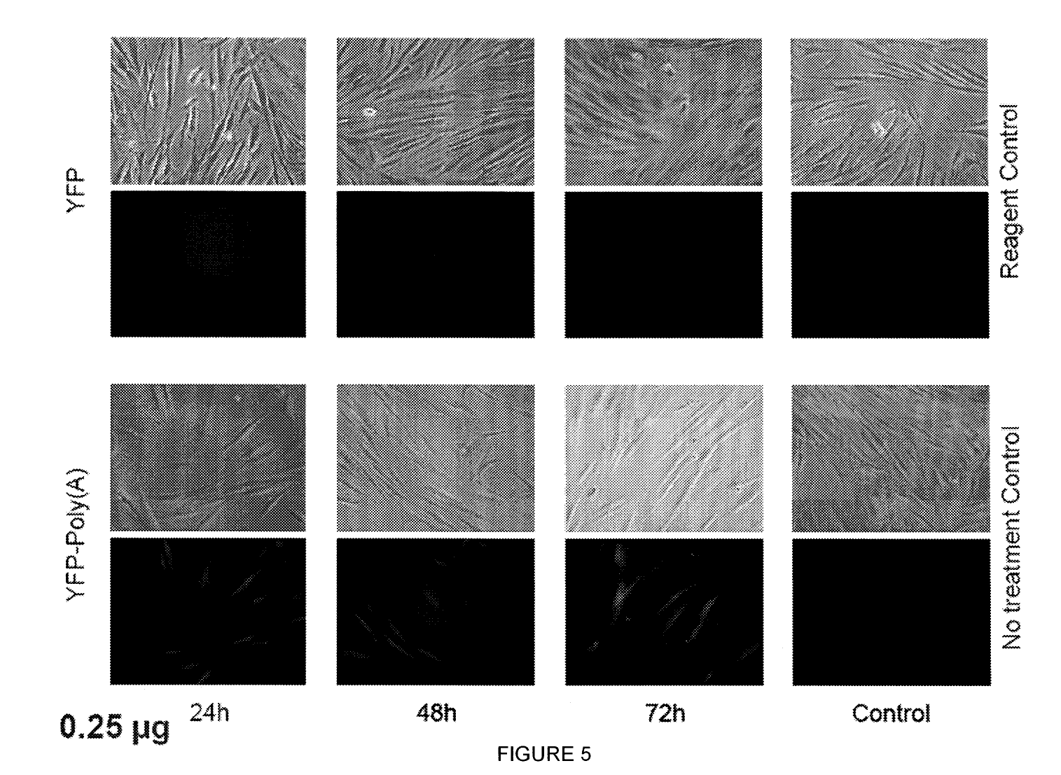

FIG. 5 also contains the results of the control run which reveal that these cells express YFP after mRNA lipofection.

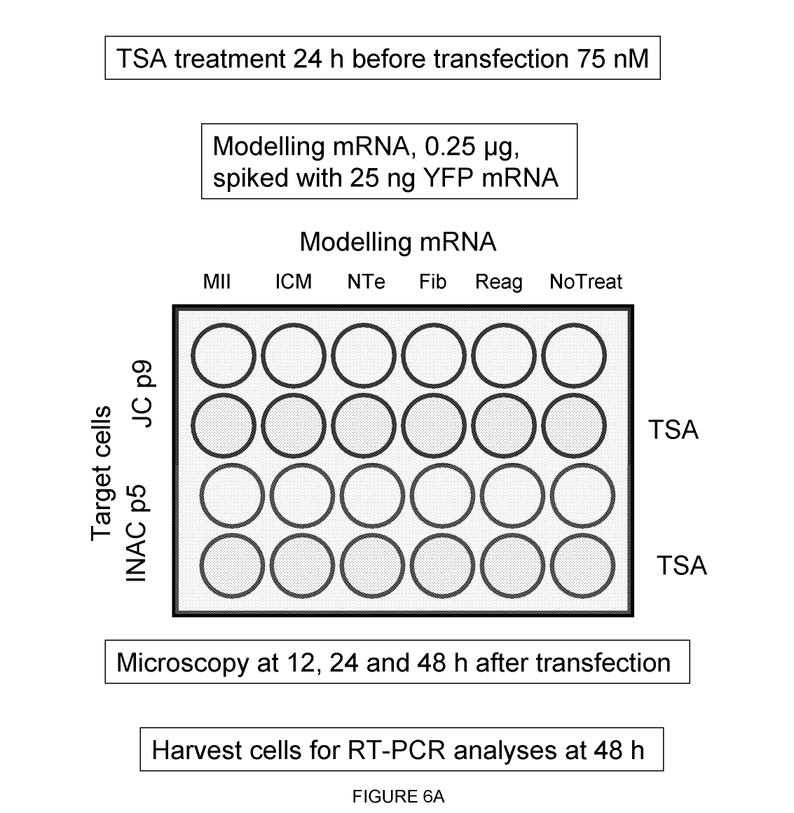

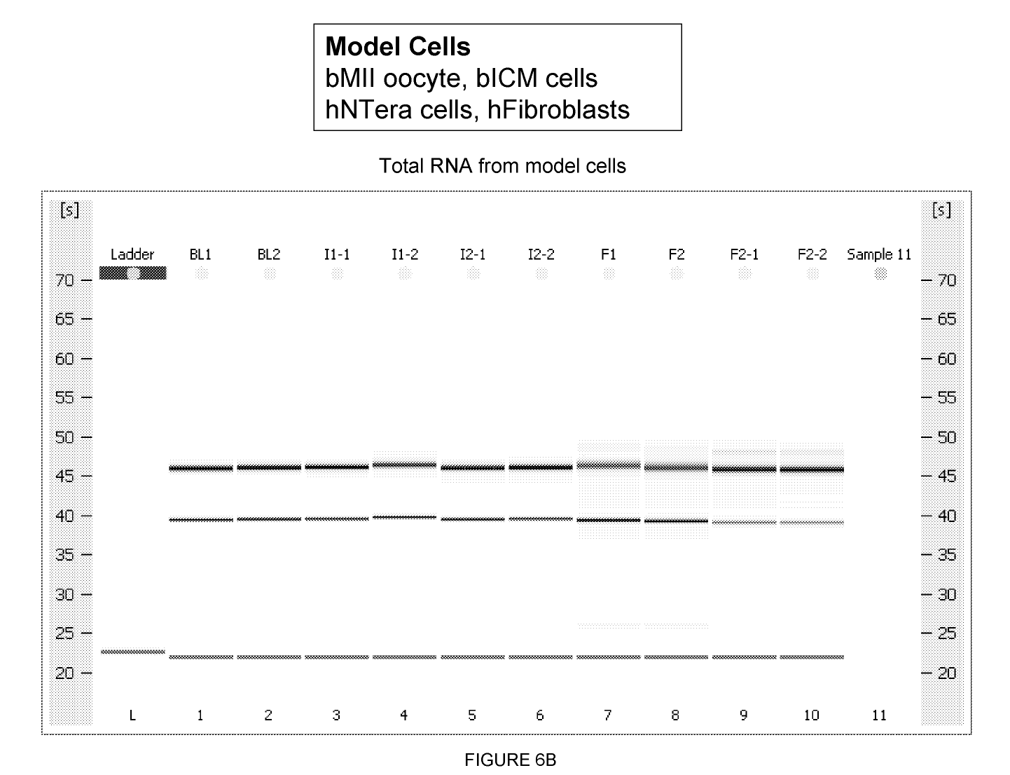

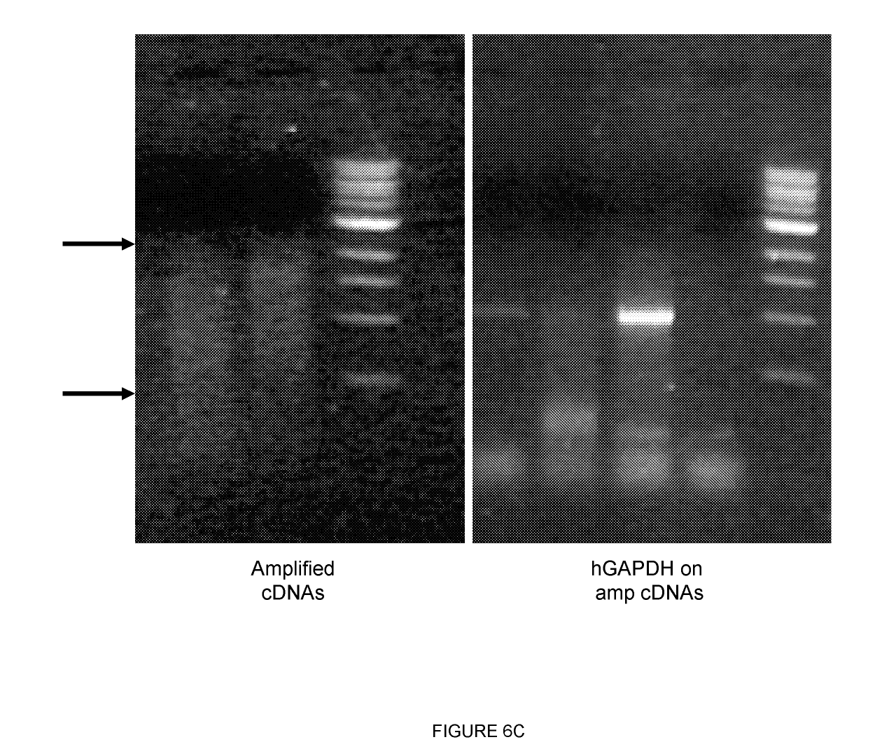

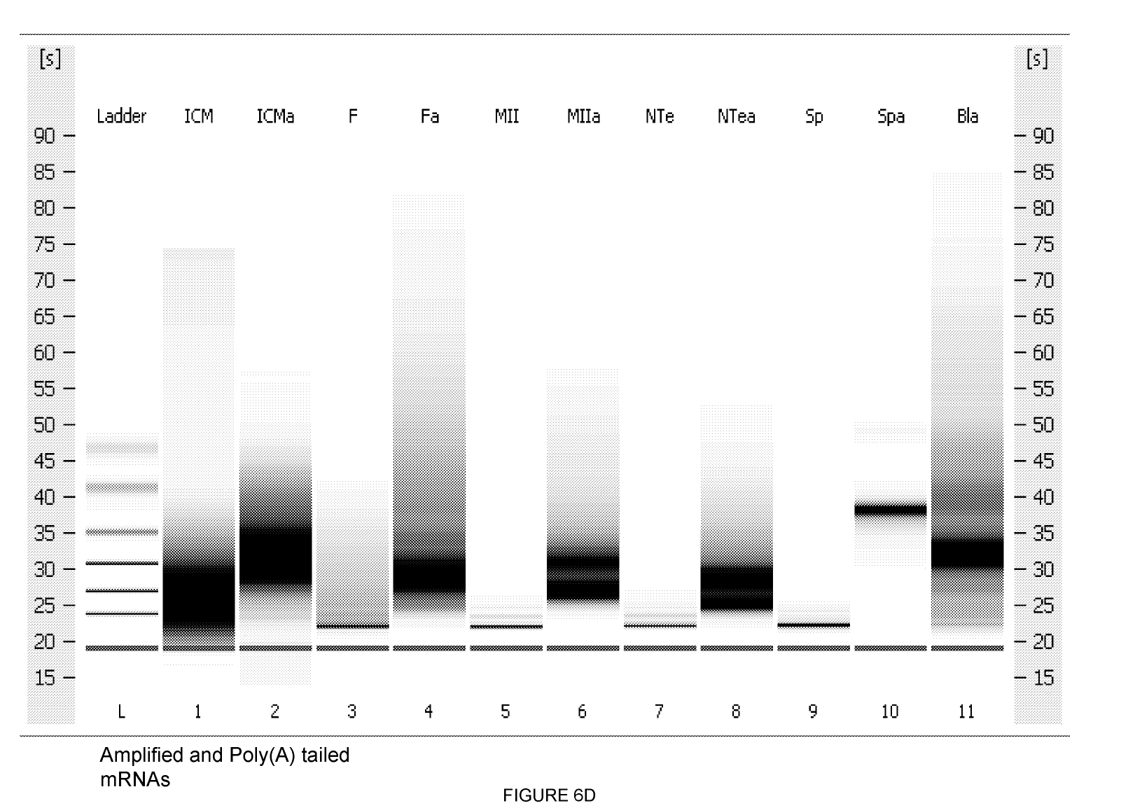

FIG. 6 shows that mRNA from oocytes, inner cell mass (ICM) of blastocysts, Ntera cells or fibroblasts themselves (the target cells in this particular experiment) can be isolated and amplified. On the right side of the figure is shown that in the experimental protocol TSA (trichostatine) may be used to facilitate reprogramming. The target cells are fibroblasts from an adult human (JC and INAC).

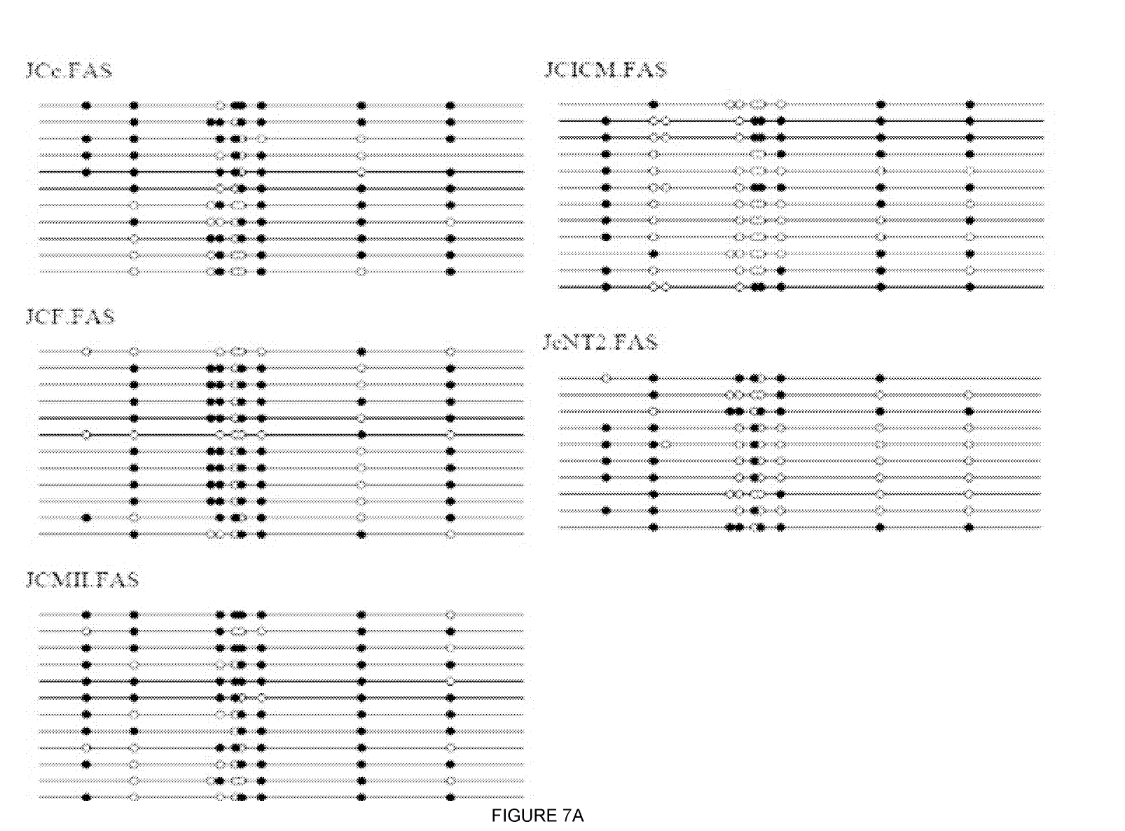

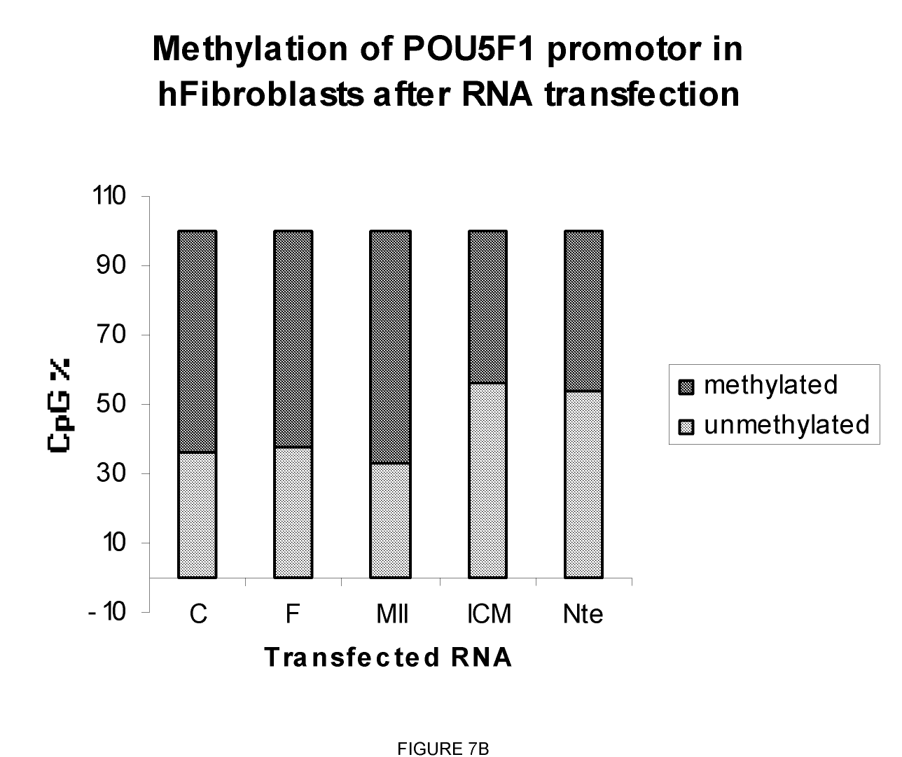

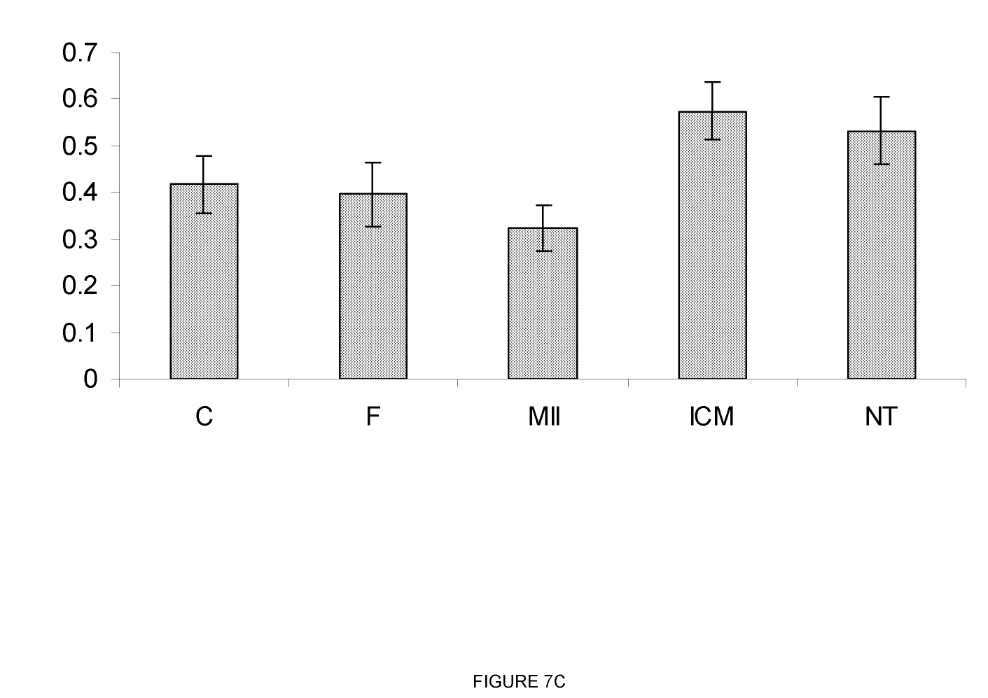

FIG. 7 contains results substantiating the fact that the inventive mRNA dedifferentiation protocol can be used to reprogram cells into a different phenotype. A key piece of evidence that reprogramming has occurred is experimental data indicating that the promoter region of genes such as Oct4 (POU5F1) is demethylated (consistent with pluripotent cells). Herein it is shown that the cells into which were introduced RNA from pluripotent cells (ICM and Ntera cells) have a lower level of methylation in that region of the promoter.

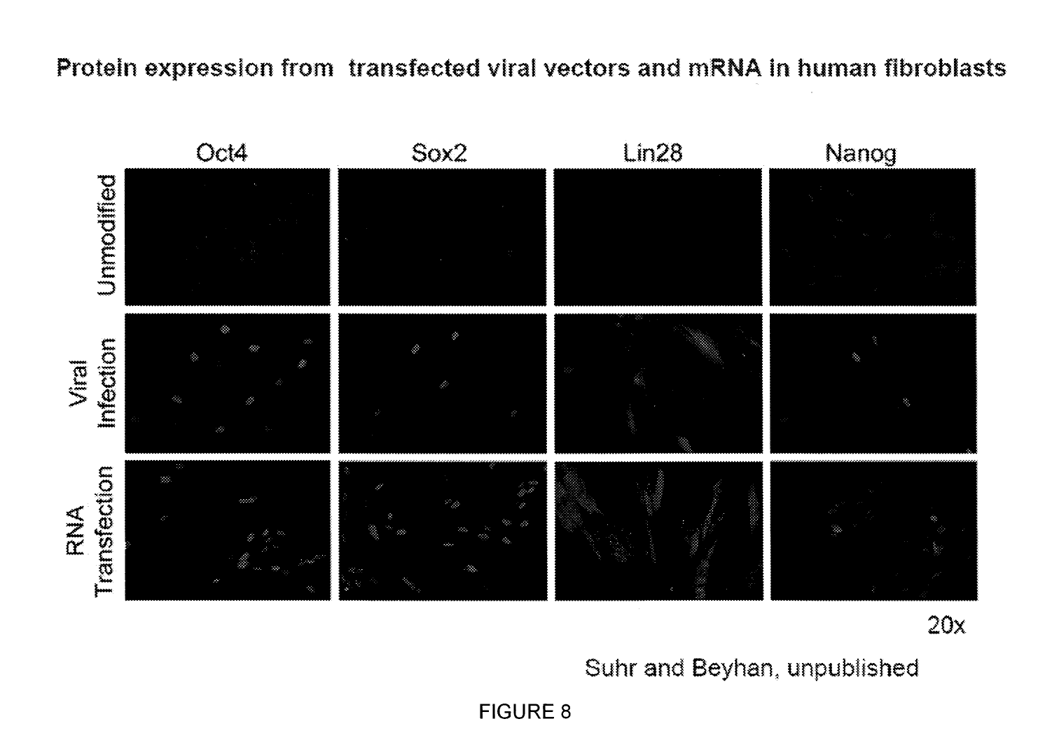

FIG. 8 shows that when mRNA from specific transcription factors is injected into target cells such as Oct4, Sox2, Lin28 and nanog, that protein transcription results in the target cells. The same figure also contains the results of experiments that reveal that viral infection instead of RNA transfection results in less protein production.

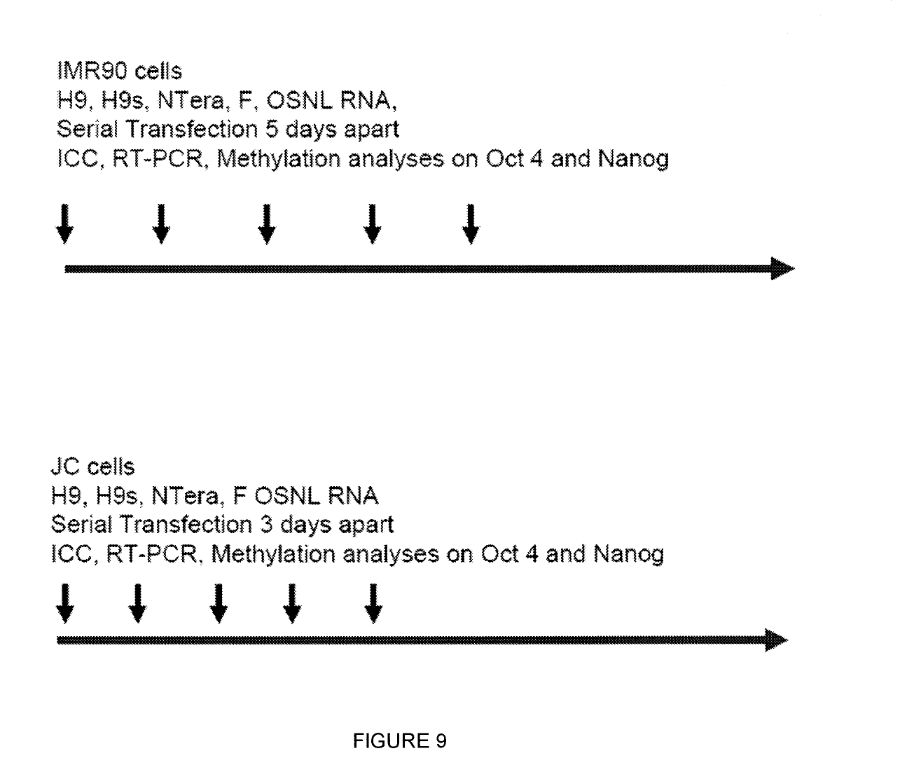

FIG. 9 contains a timeline of the experiments depicted in the Figures.

### DETAILED DESCRIPTION OF THE INVENTION

This invention describes mean for changing the phenotype or fate of cells. By using epigenetic modifications, the present invention can dedifferentiate or transdifferentiate cells of a recipient, e.g., an individual in need of cell or gene therapy. This invention solves the problem of immunorejection as cells from one patient can be transformed into a different type of cell thereby allowing for the production or creation of specific types of cells needed for the treatment of a particular disease the patient may be suffering from, e.g., pancreatic islet cells for the treatment of diabetes or hepatocytes for the treatment of liver disease. Also, this invention provides for the formation of donor compatible pluripotent cells, e.g., stem cells thereby allowing for the derivation of different somatic cell phenotypes therefrom. In addition, while the cells produced according to the invention are especially desired for cell therapy they may also be used for study of mechanisms involved in cell differentiation and disease progression.

This invention therefore addresses the lack of immuno-compatible human cells/tissues for transplantation based therapies. The inventive method will allow the generation of cells that are fully compatible with the patient by the transfer of total RNA from one cell type (donor) into that of a recipient cell, e.g., a human fibroblast or keratinocyte or white blood cell or other cell which is readily available, easily isolated and expandable in culture. For example, the invention would obtain a simple skin biopsy and isolate primary fibroblasts (or any other cell that is easy to obtain e.g. white blood cells, Keratinocytes, etc), optionally expand them in vitro and later transdifferentiate or dedifferentiate them into desired cell populations by RNA transfection. For example if these recipient somatic cells are to be converted into pluripotent cells RNA would be isolated from embryonic stem cells, human or non-human PGC's, human or non-human teratocarcinoma cells, preimplantation embryos, or oocytes from human or non-human sources and used to convert these somatic cells into a less dedifferentiated state, ideally into pluripotent cells which may be used to derive different human cell lineages.

By contrast, if these human somatic cells are to be transdifferentiated into another cell lineage, e.g. hepatocytes, beta cells, et al then total RNA would be isolated from the desired cell type, e.g., human hepatocytes and then introduced into the recipient or target cells. These cells are then incubated or cultured under conditions whereby the donor RNA converts the somatic cell of one lineage (fibroblast) into a different cell lineage such as a hepatocyte or beta cell or another cell that is desired for cell therapy.

Examples of donor cells from which RNA or mRNA can be taken to achieve pluripotency in the ‘target’ cells include by way of example: Human and/or Mouse Embryonic Stem cell, Human and/or Mouse Primordial Germ Cells, Mouse Teratocarcinoma cells, Mouse Embryonic-carcinoma cells, preimplantation embryos and oocytes from any species including human and vertebrates such as amphibians, fish, and mammals.

Examples of recipient or target cells into which RNA or mRNA can be introduced to achieve pluripotency or transdifferentiation in the ‘target’ cells include by way of example primary fibroblasts, Keratinocytes, white blood cells and other cells which are easily isolated and which ideally may be expanded and maintained in culture for prolonged time periods.

Examples of somatic cells which may be used as the donor cell for transdifferentiation include any cell type that is desired for cell therapies including by way of example hepatocytes, lymphocytes, beta cells, neural cells, cardiac cells, lung cells, etc.

Essentially, and as shown schematically in FIG. 1 the present invention effects dedifferentiation of target cells using total RNA or mRNA. The mRNA or total RNA used to effect dedifferentiation is preferably isolated from cells that are either pluripotent or which are capable of turning into pluripotent cells (oocyte). Examples thereof include by way of example Ntera cells, human or other ES cells, primordial germ cells, and blastocysts. Alternatively the RNA used to effect dedifferentiation may comprise mRNA encoding specific transcription factors. This RNA is desirably amplified using methods shown in FIGS. 2 and 3.

The total RNA or mRNA's my be e delivered into target cells by different methods including e.g., electroporation, liposomes, and mRNA injection. As shown in FIG. 1 the target cells into which RNA's are introduced and which are to be dedifferentiated according to the invention are cultured in a medium containing one or more constituents that facilitates transformation of cell phenotype. These constituents include by way of example epigenetic modifiers such as DNA demethylating agents, HDAC inhibitors, histone modifiers; and cell cycle manipulation and pluripotent or tissue specific promoting agents such as helper cells which promote growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, procarbazine, et al. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Preferred examples of histone modifiers include PARP, the human enhancer of zeste, valproic acid, and trichostatine. Particular constituents that the inventors utilize in a preferred media in order to facilitate RNA transformation and dedifferentiation of the RNA comprising target cells into pluripotent cells include trichostatine, valproic acid, zebularine and 5-aza.

As shown schematically in FIG. 1 these target cells into which RNA is introduced are cultured for a sufficient time in media that promotes RNA transformation until dedifferentiated cells (pluripotent) cells are obtained. As shown in FIG. 1 the resultant dedifferentiated cells are used to produce desired cell types or remodeled cells which may be used for transplantation, for use in animal models such as animal disease models or animal models used in the study of potential therapeutics or these may be employed in in vitro models, e.g. in studies of factors or conditions which promote the differentiation of pluripotent cells into desired cell lineages.

In a preferred embodiment the present invention introduces total RNA or mRNA's from one cell type such as a pluripotent or somatic cell into a desired human somatic cell such as a fibroblast in order to dedifferentiate or transdifferentiate such cell into a pluripotent cell or a different somatic cell corresponding to the lineage of the cell from which the donor total RNA is derived. This may be sufficient to effect cell dedifferentiation or transdifferentiation. In some instances this methodology may be combined with other methods and treatments involved in the epigenetic status of the recipient or target cell such as the exposure to DNA and histone demethylating agents, histone deacetylase inhibitors, histone modifiers, etc.

In addition, the recipient cells may be cultured under different conditions that enhance reprogramming efficiency such as co-culture of the RNA transfected cells with other cell types, conditioned medias, and by the supplementation of the culture medium with other biological agents such as growth factors, hormones, vitamins, etc. which enhance growth and maintenance of the cultured cells.

This invention therefore describes a method of changing the fate or phenotype of cells. By using epigenetic modifications, the subject methods can dedifferentiate or transdifferentiate cells. This invention is aimed to solve the problem of immuno-rejection which is evident when incompatible cells/tissues are used for transplantation. Cells from one patient can be transformed into a different type of cell allowing for the derivation of cells needed for the treatment of a particular disease the patient is suffering from. One of the types of cells that can be produced by this invention is pluripotent stem cells. This invention also offers an opportunity to the research community to study the mechanisms involved in cell differentiation and disease progression.

In addition, the invention provides novel and improved cell and gene therapies using the transdifferentiated and dedifferentiated somatic cells produced by the present invention.

The transdifferentiated or dedifferentiated or reprogrammed cells produced according to the RNA transfer method of the invention may be used e.g., for cell therapy or for study of the differentiation process. Diseases treatable by cell therapy include by way of example cancer, autoimmunity, allergy, inflammatory conditions, infection. Cancers treatable by use of cell therapy include solid and non-solid tumor associated cancers and include by way of example hematological cancers such as myeloma, lymphoma, leukemia; sarcomas, melanomas, lung cancers, pancreatic, neurological cancers such as neuroblastomas, stomach, colon, liver, gall bladder, esophageal, tracheal, head and neck, cancers of the tongue and lip, ovarian, breast, cervical, prostate, testicular, bone and other cancers. In addition cell therapy is useful in alleviating the effects of specific treatments such as radiation and chemotherapy which may deplete specific cells such as bone marrow. Further the subject cell therapy may be used for treating infectious disease such as viral or bacterial or parasite associated diseases such as HIV. Also the subject cell therapy may be used in treating autoimmune conditions wherein the host autoimmune reaction may result in killing or depletion of host cells such as immune cells or other essential cell.

Reprogrammed or dedifferentiated or transdifferentiated cells generated from these methods may be used to replace cells in a mammal in need of a particular cell type. These methods may be used to either directly produce cells of the desired cell type or to produce undifferentiated cells which may be subsequently differentiated into the desired cell type. For example, stem cells may be differentiated in vitro by culturing them under the appropriate conditions or differentiated in vivo after administration to an appropriate region in a mammal. To optimize phenotypic and functional changes, reprogrammed cells can be transplanted into the organ (e.g., a heart) where they are intended to function in an animal model or in human patients shortly after dedifferentiation or transdifferentiation (e.g., after 1, 2, 3, 5, 7, or more days). The resultant cells implanted in an organ may be reprogrammed to a greater extent than cells grown in culture prior to transplantation. Cells implanted in an animal organ may be removed from the organ and transplanted into a recipient mammal such as a human, or the animal organ may be transplanted into the recipient.

To increase the length of time the cell may be reprogrammed in vitro prior to administration to a mammal for the treatment of disease, the donor cell may be optionally modified by the transient transfection of a plasmid containing an oncogene flanked by loxP sites for the Cre recombinase and containing a nucleic acid encoding the Cre recombinase under the control of an inducible promoter (Cheng et al., Nucleic Acids Res. 28(24):E108, 2000). The insertion of this plasmid results in the controlled immortalization of the cell. After the cell is reprogrammed into the desired cell-type and is ready to be administered to a mammal, the loxP-oncogene-loxP cassette may be removed from the plasmid by the induction of the Cre recombinase which causes site-specific recombination and loss of the cassette from the plasmid. Due to the removal of the cassette containing the oncogene, the cell is no longer immortalized and may be administered to the mammal without causing the formation of a cancerous tumor.

Examples of medical applications for these cells include the administration of neuronal cells to an appropriate area in the human nervous system to treat, prevent, or stabilize a neurological disease such as Alzheimer's disease, Parkinson's disease, Huntington's disease, or ALS; or a spinal cord injury. In particular, degenerating or injured neuronal cells may be replaced by the corresponding cells from a mammal. This transplantation method may also be used to treat, prevent, or stabilize autoimmune diseases including, but not limited to, insulin dependent diabetes mellitus, rheumatoid arthritis, pemphigus vulgaris, multiple sclerosis, and myasthenia gravis. In these procedures, the cells that are attacked by the recipient's own immune system may be replaced by transplanted cells. In particular, insulin-producing cells may be administered to the mammal for the treatment or prevention of diabetes, or oligodendroglial precursor cells may be transplanted for the treatment or prevention of multiple sclerosis. For the treatment or prevention of endocrine conditions, reprogrammed cells that produce a hormone, such as a growth factor, thyroid hormone, thyroid-stimulating hormone, parathyroid hormone, steroid, serotonin, epinephrine, or norepinephrine may be administered to a mammal. Additionally, reprogrammed epithelial cells may be administered to repair damage to the lining of a body cavity or organ, such as a lung, gut, exocrine gland, or urogenital tract. It is also contemplated that reprogrammed cells may be administered to a mammal to treat damage or deficiency of cells in an organ, muscle, or other body structure or to form an organ, muscle, or other body structure. Desirable organs include the bladder, brain, nervous tissue, esophagus, fallopian tube, heart, pancreas, intestines, gallbladder, kidney, liver, lung, ovaries, prostate, spinal cord, spleen, stomach, testes, thymus, thyroid, trachea, ureter, urethra, and uterus. Also, these cells may also be combined with a matrix to form a tissue or organ in vitro or in vivo that may be used to repair or replace a tissue or organ in a recipient mammal. For example, reprogrammed cells may be cultured in vitro in the presence of a matrix to produce a tissue or organ of the urogenital system, such as the bladder, clitoris, corpus cavermosum, kidney, testis, ureter, uretal valve, or urethra, which may then be transplanted into a mammal (Atala, Curr. Opin. Urol. 9(6):517-526, 1999). In another transplant application, synthetic blood vessels are formed in vitro by culturing reprogrammed cells in the presence of an appropriate matrix, and then the vessels are transplanted into a mammal for the treatment or prevention of a cardiovascular or circulatory condition. For the generation of donor cartilage or bone tissue, reprogrammed cells such as chondrocytes or osteocytes are cultured in vitro in the presence of a matrix under conditions that allow the formation of cartilage or bone, and then the matrix containing the donor tissue is administered to a mammal. Alternatively, a mixture of the cells and a matrix may be administered to a mammal for the formation of the desired tissue in vivo. Preferably, the cells are attached to the surface of the matrix or encapsulated by the matrix. Examples of matrices that may be used for the formation of donor tissues or organs include collagen matrices, carbon fibers, polyvinyl alcohol sponges, acrylateamide sponges, fibrin-thrombin gels, hyaluronic acid-based polymers, and synthetic polymer matrices containing polyanhydride, polyorthoester, polyglycolic acid, or a combination thereof (see, for example, U.S. Pat. Nos. 4,846,835; 4,642,120; 5,786,217; and 5,041,138).

Additionally, these dedifferentiated somatic cells may be used to produce artificial tissues and organs by culturing said dedifferentiated cells in vitro e.g., in cell culture apparatus that are designed to facilitate the formation of desired cell structure and morphology. Additionally, these cells may be introduced into non-human animals as xenografted cells for example by injecting the dedifferentiated into desired organs. For example, dedifferentiated cells may be used to study the effect of dedifferentiated cardiac cells on damaged heart tissue to determine whether these cells promote the healing or regeneration process. Alternatively, dedifferentiated immune cells may be introduced into immunodeficient animals to assess whether this results in restoration of immune function.

In order to describe the invention in greater detail the following experimental examples and results are provided below.

### EXPERIMENTAL EXAMPLES

The Materials and Methods below may be used to isolate and amplify sense mRNA from various cell types, modify them by capping and polyadenylate so that they can be effectively used in RNA transfection experiments. This protocol provides the means for producing and transferring translation competent mRNA from one biologic sample to another with the purpose of inducing phenotypical changes in the receiving cell without introducing foreign DNA or chromosomes.

Materials and Methods

Equipment

Micro centrifuge

Nuclease free pipettes, pipette tips, microcentrifuge tubes

Thermocycler

Reagents

Smart mRNA Amplification Kit (Clontech 635001)

10 μl SMART T7 Oligonucleotide (10 μM)

5′-ACTCTAATACGACTCACTATAGGGAGAGGGCGGG-3′

10 μl cDNA Synthesis (CDS) Primer II A (10 μM)

5′-AAGCAGTGGTATCAACGCAGAGTACT(30)VN-3′

(N=A, C, G, or T; V=A, G, or C)

200 μl 5× First-Strand Buffer

20 μl DTT (100 mM)

50 μ50× dNTP Mix (10 mM each dATP, dGTP, dCTP, and dTTP)

30 μl T7 Extension Primer (10 μM)

5′-GCTCTAATACGACTCACTATAGG-3′

10 μl RNase H (10 U/μl)

200 μl 10× T7 Transcription Buffer

Anti-Reverse Cap Analog (ARCA) (Ambion AM8045)

Ultrapure Ntp Set 100 nM (GE Healthcare, 27-2025-01)

10 μl T7 RNA Polymerase (1,000 U/μl)

5 μl Control Total RNA (Human Placenta, 1 μg/μl)

ZB SMR Forward Primer (10 mM)

5 GCTCTAATACGACTCACTATAGG 3 (10 μM)

ZB SMR Reverse Primer

5 AAGCAGTGGTATCAACGCAGAGT 3 (10 μM)

NucleoSpin RNA II Purification Kit (Clontech 635990)

Atlas NucleoSpin Extract II Kit (Clontech 636971, 636972, 636973)

MMLV Reverse Transcriptase (Clontech 50298)

Advantage 2 PCR Kit (Clontech 639206 or 639207)

RNase Inhibitor (20 U/μl) (Clontech 50299)

Ethanol

T4 Gene 32 Protein (New England Biolabs M0300S)

Poly(A) Tailing Kit Ambion, 1350)

E-PAP (2 units/μl)

5× E-PAP Buffer

ATP Solution (10 mM)

25 mM MnCl2

10 μl Control DNA Template (0.5 μg/μl)

Nuclease-free water

TransIT-mRNA Transfection Kit (Mirus, MIR 2225)

mRNA Boost Reagent

TransIT-mRNA Reagent

First Strand cDNA Synthesis

Use total RNA isolated by using PicoPure RNA Isolation Kit

Preheat a thermal cycler to 70° C.

For each sample and control, combine the following reagents in a sterile 0.5 ml reaction tube:

| | |
|---|---|
| RNA sample (0.1-5 μg) | 3.00 μl |
| CDS Primer II A (10 μM) | 1.00 μl |
| Total volume | 4.0 μl |

Mix contents and spin the tube briefly in a microcentrifuge.

Incubate the tube at 70° C. for 3 min, then reduce the temperature to 42° C. for 2 min.

Prepare a Master Mix for all reaction tubes

Combine the following components in the order shown:

Per reaction;

| | |
|---|---|
| 5X First-Strand Buffer | 2.00 μl |
| DTT (100 mM) | 0.50 μl |
| RNase inhibitor | 0.25 μl |
| SMARTT7 Oligo (10 μM) | 1.00 μl |
| 50X dNTP Mix (10 mM each) | 1.00 μl |
| T4 G32 Protein | 0.25 μl |
| MMLV Reverse Transcriptase | 1.00 μl |
| Total volume | 6.00 μl |

Add enzyme to Master Mix just prior to use.

Mix well by vortexing and spin the tube briefly in a microcentrifuge.

Aliquot 6.0 μl of the Master Mix into each reaction tube. Use a fresh pipet tip for each tube, and mix thoroughly by pipetting. Spin the tube briefly in a microcentrifuge. Immediately return tube to the thermal cycler.

Incubate all reaction tubes at 42° C. for 1.5 hr.

Terminate the reaction by heating at 68° C. for 10 min.

Primer Extension

Prepare a Master Mix for all reaction tubes, plus one additional tube. Combine the following components in the order shown:

Per reaction;

| | | |
|---|---|---|
| Deionized H2O | 73.00 | μl |
| 10X Advantage2 PCR Buffer | 10.00 | μl |
| 50X dNTP Mix (10 mM each) | 2.00 | μl |
| T7 Extension Primer (10 μM) | 2.00 | μl |
| RNase H (10 U/μl) | 1.00 | μl |
| 50X Advantage2 Polymerase Mix | 2.00 | μl |
| Total volume | 90.00 | μl |

Mix well by vortexing and spin the tube briefly in a microcentrifuge.

Aliquot 90 μl of the Master Mix into each labeled 0.5 ml reaction tube of the first strand reaction

Cap the tube, and place it in the preheated thermal cycler.

Run the following thermal cycling program;

37° C. 15 min

95° C. 2 min

60° C. 1 min

68° C. 10 min

Proceed to Purification of double-stranded cDNA.

Nucleospin purification

Use the Atlas NucleoSpiN Extract II Kit and follow the procedure described below to purify your double-stranded cDNA prior to performing in vitro transcription.

Mix 2 volumes of buffer NT with 1 volume of sample (200 μl NT and 100 μl PCR reaction mix).

Place a NucleoSpin® Extract II column into a 2 ml collecting tube and load the sample. Centrifuge for 1 min at 11,000×g. Discard flow-through and place the NucleoSpin® Extract II column back into the collecting tube.

Add 600 μl buffer NT3. Centrifuge for 1 min at 11,000×g. Discard flow-through and place the NucleoSpin® Extract II column back into the collecting tube.

Centrifuge for 2 min at 11,000×g to remove buffer NT3 quantitatively. Make sure the spin column doesn't come in contact with the flow-through while removing it from the centrifuge and the collecting tube.

Place the NucleoSpin® column in a clean 1.5 ml microcentrifuge tube. Add 25 μl of Buffer NE, and let stand 1 min. Centrifuge at maximum speed for 1 min.

Repeat the elution step twice using the same elute

cDNA Amplification by LD PCR

Preheat the PCR thermal cycler to 95° C.

For each reaction, Use 41 μl of each first-strand cDNA (fill up to 41 microl with water if needed) into a labeled 0.5-ml thin wall reaction tube.

Prepare a Master Mix for all reaction tubes. Combine the following components in the order shown:

Per reaction;

| | |
|---|---|
| 10X Advantage 2 PCR Buffer | 5.00 μl |
| 50X dNTP (10 mM) | 1.00 μl |
| ZB SMRT Forward Primer (10 mM) | 1.00 μl |
| ZB SMRT Reverse Primer (10 mM) | 1.00 μl |
| 50X Advantage 2 Polymerase Mix | 1.00 μl |
| Total volume: | 9.00 μl |

Mix well by vortexing and spin the tube briefly in a microcentrifuge.

Aliquot 9 μl 1 of the PCR Master Mix into each tube and Mix well by vortexing and spin the tube briefly in a microcentrifuge.

Cap the tube, and place it in the preheated thermal cycler.

Run the following thermal cycling program:

95° C. for 1 min

94° C. for 30 sec

62° C. for 30 sec

68° C. for 10 min

16 cycles (number of cycles should be optimized depending on your starting material, if you use˜2 ng total RNA, around 20 cycles should be sufficient):

When the cycling is completed, adjust the reaction volume to 100 μl with 10 mM TRIS (pH 7.0).

Nucleospin Purification

Use the Atlas NucleoSpiN Extract II Kit and follow the procedure described below to purify your double-stranded cDNA prior to performing in vitro transcription.

Mix 2 volumes of buffer NT with 1 volume of sample (200 μl NT and 100 μl PCR reaction mix).

Place a NucleoSpin® Extract II column into a 2 ml collecting tube and load the sample. Centrifuge for 1 min at 11,000×g. Discard flow-through and place the NucleoSpin® Extract II column back into the collecting tube.

Add 600 μl buffer NT3. Centrifuge for 1 min at 11,000×g. Discard flow-through and place the NucleoSpin® Extract II column back into the collecting tube.

Centrifuge for 2 min at 11,000×g to remove buffer NT3 quantitatively. Make sure the spin column doesn't come in contact with the flow-through while removing it from the centrifuge and the collecting tube.

Place the NucleoSpin® column in a clean 1.5 ml microcentrifuge tube. Add 25 μl of Buffer NE, and let stand 1 min. Centrifuge at maximum speed for 1 min.

Repeat the elution step twice using the same elute

Quality Check Before in Vitro Transcription

Check the quality of amplified ds cDNA by PCR using a housekeeping gene.

If there is enough amount of cDNA, about 5 μl of it could be run on a gel or bioanalyzer.

Use housekeeping gene primers designed to amplify the template from the species of interest.

| | |
|---|---|
| Template-amplified cDNA | 2.00 μl |
| 10X PCR Buffer | 5.00 μl |
| 10 mM dNTP mixture | 1.00 μl |
| 50 mM MgCl2 | 1.50 μl |
| hGAPDH Forward Oligo (10 μM) | 2.00 μl |
| hGAPDH Reverse Primer (10 μM) | 2.00 μl |
| Sterile water | 36.00 μl |
| Taq DNA Polymerase (5 U/μl) | 0.50 μl |
| Total reaction volume | 50.00 μl |

Cap tubes and centrifuge briefly to collect the contents to the bottom. Incubate tubes in a thermal cycler at 94° C. for 3 minutes to completely denature the template.

Run 30 cycles of PCR amplification as follows:

94° C. for 45 sec

60° C. for 30 sec

72° C. for 50 sec

72° C. for 10 min

The samples can be stored at −20° C. until use.

Analyze the amplification products by agarose gel electrophoresis and visualize by ethidium bromide staining. Use appropriate molecular weight standards.

T7 Transcription and Capping

Prepare a rNTP mixture with Cap Analog (Since the rNTP mix in SMART mRNA Amplification Kit does not include Cap Analog do not use it)

| | |
|---|---|
| UTP | 22.50 mM |
| ATP | 22.50 mM |
| TTP | 22.50 mM |
| GTP | 4.50 mM |
| ARCA | 18.00 mM |

Prepare a Transcription Master Mix for all reaction tubes, plus one additional tube. Combine the following components at room temperature, in the order shown:

Per reaction;

| | |
|---|---|
| 10X T7 Transcription Buffer | 2.00 μl |
| 3X rNTP Mix (the one you prepared) | 7.00 μl |
| RNase inhibitor | 1.00 μl |
| T7 RNA Polymerase (1,000 U/μl) | 1.00 μl |
| Total volume | 11.00 μl |

Mix well by vortexing and spin the tube briefly in a microcentrifuge.

Aliquot 11 μl of the Transcription Master Mix into each tube of purified cDNA (9.0 μl)

Mix well by vortexing and spin the tube briefly in a microcentrifuge.

Incubate the tube at 37° C. for 12 hr.

Nucleospin RNA II Purification

To purify the mRNA from unincorporated ribonucleotides and small (<0.1 kb) cDNA and RNA fragments, use the provided NucleoSpin RNA II Kit and follow the procedure described below.

Add 300 μl of Buffer RA1 to each tube of IVT reaction. Mix well by pipetting.

Add 240 μl 100% ethanol to each tube. Mix well.

Place the NucleoSpin column (blue) in a 2 ml Collection Tube and load the sample into the column. Centrifuge at 8,000×g for 60 sec. Discard the flowthrough.

Add 750 μl of Buffer RA3 to the NucleoSpin® column. Centrifuge at 14,000×g for 1 min. Discard the flowthrough, and place the NucleoSpin® column back in the 2 ml Collection Tube.

Repeat RA3 wash two times, using 250 μl of Buffer RA3 each time.

Centrifuge at 14,000×g for 1 min to completely remove any residual wash buffer and dry the column filter. Place the NucleoSpin® column into a 1.5 ml microcentrifuge tube (provided).

Elute the RNA by adding 25 μl Nuclease-free Water, allowing the filter to soak for 2 min, and centrifuging at maximum speed for 1 min.

Repeat elution step twice by using the same elute.

If abundant amount of RNA is expected, elute in two steps using 35 μl for the first elution and 20 μl for the second elution.

After elution, discard the NucleoSpin column and centrifuge the eluted RNA at maximum speed for an additional 3 min.

Transfer the supernatant to a new tube. Estimate the yield and quality by Bioanalyzer.

Poly (A) Tailing of Amplified and Capped Sense mRNA

At room temp, add the tailing reagents in the order shown to

Per reaction;

| | |
|---|---|
| Amplified mRNA | 20.00 μl |
| Nuclease-free Water | 36.00 μl |
| 5×E-PAP Buffer | 20.00 μl |
| 25 mM MnCl2 | 10.00 μl |
| 10 mM ATP | 10.00 μl |
| Total volume | 96.00 μl |

Remove 0.5 μl of the reaction mixture before adding the E-PAP enzyme; as minus-enzyme control

Add 4 μl of E-PAP, and mix gently.

Incubate at 37° C. for 1 hour.

Place reaction on ice or store at −20° C.

Nucleospin RNA II Purification

To purify the mRNA from unincorporated ribonucleotides and small (<0.1 kb) cDNA and RNA fragments, use the provided NucleoSpin RNA II Kit and follow the procedure described below.

Add 300 μl of Buffer RA1 to each tube of IVT reaction. Mix well by pipetting.

Add 240 μl 100% ethanol to each tube. Mix well.

Place the NucleoSpin column (blue) in a 2 ml Collection Tube and load the sample into the column. Centrifuge at 8,000×g for 60 sec. Discard the flowthrough.

Add 750 μl of Buffer RA3 to the NucleoSpin® column. Centrifuge at 14,000×g for 1 min. Discard the flowthrough, and place the NucleoSpin® column back in the 2 ml Collection Tube.

Repeat RA3 wash two times, using 250 μl of Buffer RA3 each time.

Centrifuge at 14,000×g for 1 min to completely remove any residual wash buffer and dry the column filter. Place the NucleoSpin® column into a 1.5 ml microcentrifuge tube (provided).

Elute the RNA by adding 25 μl Nuclease-free Water, allowing the filter to soak for 2 min, and centrifuging at maximum speed for 1 min.

Repeat elution step twice by using the same elute.

If abundant amount of RNA is expected, elute in two steps using 35 μl for the first elution and 20 μl for the second elution.

After elution, discard the NucleoSpin column and centrifuge the eluted RNA at maximum speed for an additional 3 min.

Transfer the supernatant to a new tube. Estimate the yield and quality by Bioanalyzer.

Check the quality of PolyAdenylated mRNA using Bioanalyzer or denaturing agarose gel electrophoresis.

Transfection of mRNA into Eukaryotic Cells

Depending on the need, size of the plate needs be adjusted and the reagents and media should be scaled accordingly.

Plate the cells approximately 24 hours prior to transfection, plate cells at an appropriate cell density to obtain˜60-90% confluency the following day (3-5×104/well of 24-well plate).

Incubate the cells overnight.

Complex Formation (perform this procedure immediately prior to transfection)

This stem may need to be optimized for different cell types and RNA samples.

To a sterile plastic tube containing 50 μl of serum-free medium;

Add 0.25 μg RNA (0.5-1.5 μg) and mix thoroughly by pipetting.

Immediately add 1 μl mRNA Boost Reagent (0.5 to 2 μl) and mix thoroughly by pipetting.

Immediately add 2 μl TransIT®-mRNA Reagent (1 to 3 μl) and mix thoroughly by pipetting

Incubate at room temperature for 3 (2-5) minutes. Do not let the complexes incubate longer than 5 minutes as this may decrease transfection efficiency.

NOTE: The TransIT-mRNA Transfection Kit yields improved transfection efficiencies when transfections are performed in complete growth medium (instead of serum-free medium) with no media change post-transfection. If necessary, remove the medium from the cells and replace it with 0.5 ml per well of fresh complete growth medium.

Add the RNA/mRNA Boost Reagent/TransIT-mRNA Reagent complex mixture to the cells. Gently rock the dish back and forth and from side to side to distribute the complexes evenly. Do not swirl the plate.

Incubate for 4 to 48 hours depending on the RNA transfected and the goal of the experiment.

### EXAMPLE 1

Transdifferentiation of Human Primary Fibroblasts into Human Hepatocytes Primary fibroblasts are collected from a human skin biopsy sample by known methods. Total RNA is isolated from human hepatocytes or bovine hepatocytes using the protocol provided above. The resultant total mRNA or RNA is then packaged and is delivered into the target cells (primary fibroblasts) by use of standard transfection techniques. These cells are then maintained in culture under conditions and for a sufficient time for these cells to convert into human hepatocytes. These cells may be used for therapies wherein immunocompatible hepatocytes are therapeutically effective such as the treatment of liver disease, e.g., cirrhosis or cancer.

### EXAMPLE 2

### Dedifferentiation of Human Fibroblasts into Pluripotent Cells

Primary fibroblasts are collected from a human skin biopsy sample by known methods. These cells are optionally expanded in vitro by known culture methods. Total RNA is isolated from human embryonic stem cells or inner cell mass cells derived from 7 day old bovine embryos oocytes using the protocol provided above. The resultant total mRNA or RNA is then packaged and is delivered into the target cells (primary fibroblasts) by use of standard transfection techniques. These cells are then maintained in culture under conditions and for a sufficient time for these cells to convert into pluripotent cells. These cells may be used to derive different human cell lineages for human therapy culture which are donor immunocompatible.

### EXAMPLE 3

### Transdedifferentiation of White Blood Cells into Beta Cells

White blood cells are isolated from the blood of a human donor for which immunocompatible cells for therapy are desired. These cells are optionally expanded in vitro by use of known culture methods. Total RNA is isolated from beta cells derived from human or non-human donors using the total RNA isolation protocol provided above. The resultant total mRNA or RNA is then packaged and is delivered into the target cells (human leukocytes) by use of standard transfection techniques. These cells are then maintained in culture under conditions and for a sufficient time for these cells to convert into beta cells. These cells are used for therapies wherein these cells are therapeutically effective such as diabetes.

### EXAMPLE 4

### Transdifferentiation of Human Keratinocytes into Neural Cells

Keratinocytes are isolated from the skin of a human donor for which immunocompatible cells for therapy are desired. These cells are optionally expanded in vitro by use of known culture methods. Total RNA is isolated from neural cells derived from human or non-human donors using the total RNA isolation protocol provided above. The resultant total mRNA or RNA is then packaged and is delivered into the target cells (human keratinocytes) by use of standard transfection techniques. These cells are then maintained in culture under conditions and for a sufficient time for these cells to convert into neural cells. These cells are used for therapies wherein these immunocompatible neural cells are therapeutically effective such as Parkinson's disease or Alzheimer's disease.

### EXAMPLE 5

### Dedifferentiation of Primary Keratinocytes into Immunocompatible Pluripotent Cells

Primary keratinocytes are isolated from the skin biopsy sample of a human donor for which immunocompatible cells for therapy are desired. These cells are optionally expanded in vitro by use of known culture methods. Total RNA is isolated from pluripotent cells derived from human or non-human donors, e.g. human ESC's or inner cell mass cells from a 7 day old bovine embryo using the total RNA isolation protocol provided above. The resultant total mRNA or RNA is then packaged and is delivered into the target cells (human keratinocytes) by use of standard transfection techniques. These cells are then maintained in culture under conditions and for a sufficient time for these cells to convert into dedifferentiated cells which are pluripotent. These cells may be used to derive different immunocompatible cell lineages which can be used in cell therapies.

### EXAMPLE 6

### Transdifferentiation of Human Primary Fibroblasts into Cardiac Cells

Primary fibroblasts are isolated from the biopsied skin sample of a human donor for which immunocompatible cardiac cells for therapy are desired. These cells are optionally expanded in vitro by use of known culture methods. Total RNA is isolated from cardiac cells derived from human or non-human donors using the total RNA isolation protocol provided above. The resultant total mRNA or RNA is then packaged and is delivered into the target cells (human fibroblasts) by use of standard transfection techniques. These cells are then maintained in culture under conditions and for a sufficient time for these cells to convert into cardiac cells. These cells are used for therapies wherein these cells are therapeutically effective such as chronic heart disease or heart attack.

### EXAMPLE 7

### Control Using YFP-Poly(A) Transfected Cells

This experiment relates to a control showing that yellow fluorescent (YFP) protein can be in vitro transcribed with high quality and delivered to cells. donors using the total RNA isolation protocol provided above. As shown in FIG. 3 in this experiment a T3-YFP retroviral vector is used for in vitro transcription with cap analog Ambion, mMessage mMachine T3 kit and polyadenylated with Ambion Poly(A) tailing kit. The resultant RNAs are then introduced into target cells (JC fibroblasts) comprised in 24-well plates 24 hours after plating which cells are at 60% confluency comprised in a 500 microliters of media using Mirus, TransIT-mRNA transfection Kit. In addition, similar controls are effected using YFP, reagent control, and control (no reagent) at 25 and 50 micrograms. Fluorescence microscopy is effected to detect YFO 12, 24, 48, and 72 hours after transfection. As shown in FIG. 4 the YFP-poly(A) and YFP transfected cells express YFP based on detected fluorescence and the control and reagent control treated cells do not.

### EXAMPLE 8

### Dedifferentiation of Fibroblasts by Transfection with RNA from Pluripotent Cells (Oocyte, bICM, hNTera Cells)

This experiment relates to the experiments in FIGS. 6 and 7. These Figures depict an experiment and results thereof wherein mRNA from oocytes, inner cell mass (ICM) of blastocysts, Ntera cells or fibroblasts themselves (the target cells in this experiment) is isolated and amplified. On the right side of the FIG. 6 is shown the experimental conditions wherein TSA (trichostatin) is being used to help reprogramming of the fibroblasts by the recipient mRNA. The target cells are fibroblasts from adult human (JC and INAC). In this experiment total RNA is isolated from the model pluripotent cells, is amplified and poly(A) tailed using same methods above-identified, and the target cells are treated with 75 nM TSA prior to transfection. mRNA from the model cells (o.25 micrograms) spiked with 25 ng YFP mRNA (and control RNAs or no treatment as shown in the Figure) are used for transfection. At 12, 24 and 48 hours post-transfection microscopy is effected and at 48 hours cells are harvested for RT-PCR analyses. As shown in FIG. 7 the cells transfected with the RNA from pluripotent cells show evidence of dedifferentiation since it was seen that the promoter region of genes such as Oct4 (POU5F1) is demethylated. In the Figure it can be seen that the cells which were injected with RNA from pluripotent (ICM and Ntera) cells have a lower level of methylation in that region of the promoter, evidence of dedifferentiation and conversion into pluripotency.

### EXAMPLE 9

### Protein Expression from Transfected Viral Vectors and mRNA in Human Fibroblasts

This experiment relates to the experiments the results of which are contained in FIG. 8. Therein protein expression is compared in cells transfected by injection with mRNA from specific transcription factors such as Oct4, Sox2, Lin28 and nanog or viral infection or no treatment. The results in the Figure reveal that viral infection rather than RNA transfection results in less protein production. FIG. 9 further shows the timeline of these experiments.

### LIST OF REFERENCES

The following references are cited in this application. The contents of these references is incorporated by reference herein.

We claim:

1. A method of reprogramming, transdifferentiating or dedifferentiating a target or recipient somatic cell into a cell of a different somatic cell type or into a pluripotent or less differentiated cell by the introduction of RNA from a donor cell which is a pluripotent cell or which is a different somatic cell under transfection and culture conditions that convert the somatic cell into a pluripotent cell or into a somatic cell of a cell lineage corresponding to that of the somatic cell from which the total RNA is isolated.

2. The method of claim 1 wherein the RNA introduced into the target cell is derived from an inner cell mass cell, an oocyte, an NTera cell, a primordial germ cell or a blastocyst.

3. The method of claim 1 wherein the target cell is treated before, during or after RNA introduction with at least one DNA methylating agent, HDAC inhibitor or histone modifier or a combination thereof.

4. The method of claim 3 wherein the target cell is treated before, during or after RNA introduction with trichostatine, valproic acid, zebularine, 5-aza, or a combination thereof.

5. The method of claim 1 wherein the RNA is introduced by one of electroporation, use of liposomes, or mRNA injections.

6. The method of claim 1 wherein the introduced RNA is mRNA encoding one or more specific transcription factors.

7. The method of claim 6 wherein said transcription factors include Oct4, Sox2, Lin28, nanog or a combination thereof.

8. The method of claim 1 wherein the target cells after introduction of RNA are cultured in a media that contains one or more moieties that favor the growth of dedifferentiated or pluripotent cells.

9. The method of claim 8 wherein these moieties include helper cells, growth factors, hormones, bioactive molecules or a combination thereof.

10. The method of claim 8 wherein the media contains at least one DNA methylating agent, HDAC inhibitor or histone modifier or a combination thereof.

11. The method of claim 1 wherein the target cell is a human somatic cell.

12. The method of claim 11 wherein the cell is a fibroblast or epithelial cell.

13. The method of claim 1 wherein the RNA is RNA and/or mRNA and/or subtracted RNA.

14. The method of claim 2 wherein the RNA is packaged into a vector prior to introduction into the somatic cell.

15. The method of claim 1 wherein the target or recipient cell is a human fibroblast cell.

16. The method of claim 1 wherein the target or recipient cell is a primary somatic cell.

17. The method of claim 1 wherein the target or recipient cell is a fibroblast, keratinocyte, or white blood cell.

18. The method of claim 1 wherein the total RNA used for transfer is derived from a pluripotent cell.

19. The method of claim 18 wherein the pluripotent cell is a human or non-human cell selected from an embryonic stem cell, primordial germ cell, teratocarcinoma cell, embryonic-carcinoma cell, cell of a preimplantation embryo, and an oocyte.

20. The method of claim 18 wherein the pluripotent cell is human, rodent, amphibian, fish or mammalian.

21. The method of claim 18 wherein the pluripotent cell is a human ESC or oocyte or inner cell mass cell from a 7 day old embryo.

22. The method of claim 1 wherein a non-pluripotent somatic cell is used as the donor cell.

23. The method of claim 22 wherein the donor somatic cell is human, rodent, amphibian, fish or non-human mammalian.

24. The method of claim 22 wherein the somatic donor cell is selected from the group consisting of a hepatocyte, lymphocyte, cardiac cell, beta cell, neural cell, gastrointestinal cell, sensory cell, muscle cell, bone cell, kidney cell, lung cell, reproductive organ cell, bladder cell, immune cell, and a skin cell.

25. The method of claim 1 wherein said method results in the dedifferentiation of said somatic cell.

26. The method of claim 1 wherein said method increases the lifespan of said cell.

27. The method of claim 1 which results in the demethylation of certain genes characteristic of pluripotency.

28. The method of claim 27 wherein said genes include the Oct4 (POU5F1) promoter, telomerase, SSEA-3, SSEA-4, TRA-1-60, TRA-1-81 and alkaline phosphatase or another gene specifically expressed in pluripotent and not in non-pluripotent cells.

29. The method of claim 1 wherein said method results in the conversion of said somatic cell into a pluripotent or multipotent cell.

30. The method of claim 1 wherein the cells after introduction of the donor RNA from a pluripotent cell are screened for pluripotency based on the expression of at least one gene that is selectively expressed by pluripotent cells.

31. The method of claim 30 wherein said gene is selected from Oct4, telomerase and SSEA-3, SSEA-4, TRA-1-60, TRA-1-81 and alkaline phosphatase.

32. The method of claim 31 wherein said expression is detected using an antibody or other ligand that specifically binds to one of said polypeptides.

33. The method of claim 1 wherein the recipient cell is a human cell.

34. The method of claim 33 wherein said human cell is selected from a fibroblast, lymphocyte, endothelial cell, keratinocyte, bone cell, neural cell, heart cell, kidney cell, tooth cell, lung cell, skin cell, immune cell, stomach cell, esophageal cell, tracheal cell, liver cell, gall bladder cell, ovarian cell, urethral cell, testicular cell, red blood cell, diaphragm cell, muscle cell, a sensory cell involved in sight, hearing, taste, smell, or touch, and a pancreatic cell.

35. The method of claim 34 which converts said cell into an embryonic or adult stem cell type.

36. The method of claim 22 which converts said cell into an embryonic-like stem cell.

37. The method of claim 1 wherein the resultant transdifferentiated cell is itself suitable for cell therapy or is used to derive somatic cells which are suitable for cell therapy.

38. The method of claim 1 wherein the recipient somatic cells after RNA transfer the are screened for the expression of at least one marker characteristic of the phenotype of the donor cell.

39. The method of claim 37 wherein the donor cell is a human somatic cell selected from a fibroblast, endothelial cell, keratinocyte, bone cell, neural cell, heart cell, kidney cell, tooth cell, lung cell, skin cell, immune cell, stomach cell, liver cell, ovarian cell, urethral cell, testicular cell, red blood cell, diaphragm cell, muscle cell, sensory cell, and pancreatic cell.

40. A cell therapy method which comprises the introduction of human somatic cells of a specific lineage or phenotype into a patient for therapy wherein the method comprises administration of cells from the patient which have been transdifferentiated into somatic cells of a specific lineage or phenotype by the method of claim 1.

41. The method of claim 40 which is used to treat a condition selected from cancer, autoimmunity, infection, inflammation disorder, and an allergic condition.


SEQUENCE LISTING (5 sequences)

SEQ ID NO: 1 - DNA, 34 bp, Artificial Sequence
  SMART T7 Oligonucleotide
actctaatacgactcactatagggagagggcggg

SEQ ID NO: 2 - DNA, 28 bp, Artificial Sequence
  cDNA Synthesis (CDS) Primer II A
aagcagtggtatcaacgcagagtactnn

SEQ ID NO: 3 - DNA, 23 bp, Artificial Sequence
  T7 Extension Primer
gctctaatacgactcactatagg

SEQ ID NO: 4 - DNA, 23 bp, Artificial Sequence
  ZB SMR Forward Primer
gctctaatacgactcactatagg

SEQ ID NO: 5 - DNA, 23 bp, Artificial Sequence
  ZB SMR Reverse Primer
aagcagtggtatcaacgcagagt